(19) United States
(12) Patent Application Publication
(10) Pub. No.: US 2014/0348755 A1
(43) Pub. Date: Nov. 27, 2014

(54) TARGETED NANOPARTICLES JOINED TO REPORTER MOLECULES THROUGH MULTIPLE MECHANISMS

(71) Applicant: NPLEX LABORATORY INC., San Jose, CA (US)

(72) Inventor: Kevin C. Weng, San Jose, CA (US)

(21) Appl. No.: 14/345,462

(22) PCT Filed: Sep. 20, 2012
(86) PCT No.: PCT/US12/56388

§ 371 Date: Mar. 18, 2014

Related U.S. Application Data
(60) Provisional application No. 61/537,438, filed on Sep. 21, 2011.

(51) Int. Cl. A61K 49/00; G01N 33/569; A61K 49/18

(52) U.S. Cl. CPC A61K 49/0065; A61K 49/1818; G01N 33/56966
USPC 424/9.32; 424/9.6; 435/7.1

(57) ABSTRACT

Nanoparticles, such as liposomes etc., containing multiple reporter molecules, e.g. dyes, fluorophores, FRET pairs, semiconductor nanocrystals, fluorescent chelates, chelate complexes, coordination complexes, mass tags, Raman tags, lanthanides, enzymes, enzymatic substrates, etc., through a combination of multiple physical and/or chemical interactions are disclosed. The reporter molecules are associated with the nanoparticle in at least two different ways, i.e. through different mechanisms. The nanoparticle is targeted through a targeting agent such as an antibody, an antibody fragment, a nucleotide, a peptide, an aptamer, biotin, avidin, or a ligand. The nanoparticles are useful in bioassays or imaging where an analyte is to be detected by binding of the nanoparticle to the analyte and detection of a signal from the reporter molecules.

### CROSS-REFERENCE TO RELATED APPLICATIONS

This application claims priority from U.S. Provisional Patent Application No. 61/537,438 filed on Sep. 21, 2011, which is hereby incorporated by reference in its entirety.

### STATEMENT OF GOVERNMENTAL SUPPORT

None.

### REFERENCE TO SEQUENCE LISTING, COMPUTER PROGRAM, OR COMPACT DISK

None.

### BACKGROUND OF THE INVENTION

1. Field of the Invention

The present invention relates to the field of chemistry, nanoformulation, and molecular biology, and more particularly to lipid based nanoparticles, such as liposomes, wherein the liposomes are modified to carry reporters such as dyes, fluorophores, mass tags, chelates, elemental probes, radiolabels, isotopes, Raman tags, semiconductor nanocrystals, colloid gold, enzymes, and enzymatic substrates, and wherein the reporters are linked to or contained in the nanoparticle through various chemical and physical mechanisms, termed herein “modes of association.” The nanoparticles further contain targeting moieties such as antibodies, antibody binding portions, antibody mimetics, aptamers, receptor ligands, biotin, avidin, streptavidin, NeutrAvidin, CaptAvidin, folic acid, nucleotides, peptides, glycoproteins, etc.

2. Related Art

Presented below is background information on certain aspects of the present invention as they may relate to technical features referred to in the detailed description, but not necessarily described in detail. That is, individual parts or methods used in the present invention may be described in greater detail in the materials discussed below, which materials may provide further guidance to those skilled in the art for making or using certain aspects of the present invention as claimed. The discussion below should not be construed as an admission as to the relevance of the information to any claims herein or the prior art effect of the material described.

Multifunctional nanoparticles are increasingly used in biomedical applications such as diagnostic imaging and drug delivery. An emerging field of use is to replace or assist traditional analytical reagents to improve the analysis procedures or outcome in various assays. They may also enable new analytical methods, such as mass cytometry.^{1,2} The unique properties of nanoparticles, including large surface areas, large interior volumes, a large number of attachment points, and tunable functionality for linking and associating with multiple recognition and/or detection components, are among the main benefits that are exploited in these applications.

Examples of multifunctional nanoparticles are lipid-based nanoparticles such as micelles and liposomes, which have been explored and developed for in vivo drug delivery^{3,4} and various analytical applications.⁵⁻⁷ In particular, liposomes utilize controlled self-assembly of amphiphilic lipids that form a spherical unilamellar lipid bilayer (shell) enclosing an aqueous interior for drug or functional agents loading. Liposomes can also exist in other complex morphologies, depending on the composition of the constituents, the medium and the conditions. The common features among the practical applications in which liposomes participate are that liposomes carry a multiplicity of functional components; for example, chemotherapeutic agents in drug delivery systems, and reporters or labels or probes in analytical reagents. These reporters and labels can be present in their interior space, lipid bilayers (shell), or on the surface via conjugation or adsorption on either or both sides of the lipid bilayers.

The advantages of using targeted lipid-based nanoparticles in analytical processes partly depend on the amount of reporters and detectable labels that are loaded into or onto the nanoparticle. To further enhance the reporter signals, increasing the loading of reporters through the various mechanisms, that is, modes of association, available is described in this application.

### SPECIFIC PATENTS AND PUBLICATIONS

Rutner et al. U.S. Pat. No. 5,248,590, entitled “Surface modified liposomes,” discloses a liposome reagent encapsulating a molecule to be targeted to a body site or used as an assay reporter has a ligand and a sulfonate-containing group on the liposome surface. Preferred ligands are antibodies or antibody fragments and preferred encapsulants are enzymes or dyes.

Zeimer U.S. Pat. No. 6,140,314, entitled “Selective and non-invasive visualization or treatment of vasculature,” discloses methods which utilize fluorescent dyes and tissue-reactive substances encapsulated within heat-sensitive liposomes which are subsequently heated to release the contents thereof at a pre-determined anatomical locus.

Huang et al. U.S. Pat. No. 4,708,933, entitled “Immunoliposome assay-methods and products,” discloses a membrane lytic immunoassay. In one embodiment of this assay, an antigen is first covalently coupled with lipids and this antigen-lipid complex is mixed with a hexagonal phase forming lipid to form bilayer liposome vesicles additionally containing a self-quenching fluorescent dye. When this antigen-containing liposome is brought into contact with a solid surface coated with antibody molecules, binding occurs between the antigen and the antibody, disrupting the liposome and releasing the dye.

However, the foregoing patents do not disclose targeted liposomes with reporter molecules having multiple, different linkage chemistries (i.e. modes of association) of the reporter molecules to the liposome.

### BRIEF SUMMARY OF THE INVENTION

The following brief summary is not intended to include all features and aspects of the present invention, nor does it imply that the invention must include all features and aspects discussed in this summary.

The present invention comprises, in certain aspects, a nanoparticle comprising: (a) an organic nanoparticle having an external wall, an inner core region, and, optionally, interior wall portions defining additional inner core regions therein; (b) targeting molecules linked to said external wall; and (c) reporter molecules associated with said nanoparticle through at least two different mechanisms.

The different mechanisms of reporter molecule linkages may be selected from at least two of: (i) encapsulation of reporter molecules in one or more inner core regions; (ii) embedding of reporter molecules in one or more of said external wall and said interior wall portions; (iii) chemical linkage of reporter molecules to one or more of said external wall and said interior wall portions; (iv) specific binding of reporter molecules to one or more of said external wall and said interior wall portions through binding partners that are linked to the wall portions; and (v) electrostatic binding of reporter molecules to one or more of said external wall and said interior wall portions.

In certain aspects of the invention, the nanoparticle may be selected from the group comprising a liposome, a micelle, a multilamellar vesicle, or a multi-vesicular vesicle.

In certain aspects of the invention, the external wall of the nanoparticle comprises lipids and lipophilic components. These may include one or more of glycerolipids, phosphatidic acids, phosphatidyl choline, phosphatidyl ethanolamine, phosphatidyl serine, phosphatidyl glycerol, phosphatidyl inositol, sphingolipids, ceramides, sterol lipids, functionalized lipids, cross-linked lipids, and PEGylated lipids

In certain aspects, the reporter molecule of the nanoparticle composition is a single species of dye or more than a single species of dye. The dyes may be of a single species or more than one species. The dye(s) may include at least one of fluorescein, rhodamine, coumarin, BODIPY, Cascade Blue, Pacific Blue, Pacific Orange, NBD, Lucifer Yellow, phycobiliprotein, Texas Red, cyanine, Alexa Fluor, eFluor, DyLight Fluor, or their derivatives.

In certain aspects, the reporter molecule of the nanoparticle composition is a chelating agent, a metal, a chelation complex, a coordination complex, a lanthanide complex, or a metal complex. In one embodiment, the chelate is a lanthanide chelate. In other embodiments, the reporter molecule selected is a semiconductor nanocrystal, an enzyme, an enzymatic substrate, a mass tag, or a Raman tag.

In certain aspects, the targeting molecule is at least one member selected from the group consisting of an antibody, antibody fragment, antibody mimetic, peptide, nucleotide, aptamer, sugar, glycoprotein, biotin, avidin, streptavidin, NeutrAvidin, CaptAvidin, or folic acid. The targeting molecule may be linked to the external wall through crosslinking of maleimide and sulfhydryl groups, or crosslinking of amine and carboxyl groups.

In certain aspects, the nanoparticle composition comprises a targeting molecule where the targeting molecule is an antibody or antibody fragment linked to the external wall and the reporter molecule is a fluorescent dye encapsulated within the interior region, embedded in the external wall, conjugated on the internal or external, either or both sides of the walls of the nanoparticle.

In certain aspects, the nanoparticle composition comprises a targeting molecule which is an antibody or antibody fragment linked to molecules comprising the external wall and the reporter molecule is a metal chelate both encapsulated within the interior region and conjugated on the internal or external wall, either on one or both sides of the walls of the nanoparticle.

In certain aspects, the present invention comprises a method of detecting an analyte in a test solution. A nanoparticle composition as described above is added to the test solution. Detection of the presence of nanoparticles bound to the analyte by the targeting molecule is accomplished by detecting a signal from the reporter molecule. Detection may comprise the methods of immunoassays, immunohistochemistry, immunocytochemistry, Western blotting, dot blotting, flow cytometry, fluorescent activated cell sorting (FACS), bead assays, ELISA, microarrays, capillary electrophoresis, multiplex analysis, chromatography, sensors and microfluidic systems.

In certain aspects, the present invention also comprises a method of detecting an antigen on a tissue or a molecular target biological sample comprising contacting said tissue or biological sample with a nanoparticle composition as described above and detecting the presence of nanoparticles bound to the tissue or biological sample by the targeting molecule by detecting a signal from the reporter molecule.

In certain aspects, the present invention relates to a method of imaging or delivering specific effects to biological samples, tissues, organs, animals, humans comprising adding to an imaged or treated subject a nanoparticle composition as described above and detecting the presence of nanoparticles by detecting a signal from the reporter molecule or sensitizing the nanoparticles by providing stimulation to the labels.

In certain aspects, the method for detecting as described above comprises magnetic resonance imaging, positron emission tomography, photo acoustic imaging, computed tomography, single-photon emission computed tomography, radio-sensitization, and photo-sensitization.

In certain aspects, the present invention further comprises a method for preparing an organic nanoparticle. The method comprising the steps of: (a) preparing an organic nanoparticle having an external wall, an inner core region, and, optionally, interior wall portions defining therein additional inner core regions; (b) attaching a targeting molecule linked to said external wall by coupling said targeting molecule to the external wall; and (c) incorporating reporter molecules to said nanoparticle through at least two different methods. Such methods consist of: (i) encapsulation of reporter molecules in one or more inner core regions; (ii) embedding of reporter molecules in one or more of said external wall and said interior wall portions; (iii) chemical linkage of reporter molecules to one or more of said external wall and said interior wall portions; (iv) specific binding of reporter molecules to one or more of said external wall and said interior wall portions through binding partners; and (v) electrostatic binding of reporter molecules to one or more of said external wall and said interior wall portions, wherein steps (a), (b), and (c) (i) through (c) (v) may be carried out in various orders.

In certain aspects, step (a) of the above method comprises preparing a liposome, a micelle, a multilamellar vesicle, or a multi-vesicular vesicle. Further, the embedding of reporter molecules in step (c) (ii) may be carried out with a lipophilic dye.

In certain aspects, the targeting molecule is an antibody or antibody fragment lined to said external wall. The reporter molecules in the above methods are selected from the group consisting of a chelate complex, a coordination complex, lanthanide chelate, a semiconductor nanocrystal, an enzyme, an enzymatic substrate, a mass tag, or a Raman tag. The reporter molecules may comprise self-quenching fluorophores, or fluorescence resonance energy transfer (FRET) pairs.

In certain aspects, the above method may further comprise the step of linking polyethylene glycol molecules to the nanoparticle.

Certain aspects of the present invention comprise a method of detecting an analyte in a biological sample or test solution comprising adding to the biological sample or test solution a nanoparticle composition as described above and detecting the presence of nanoparticles bound to the analyte by the targeting molecule by detecting a signal from the reporter molecule.

Such analytical methods may include the use of an immunoassay, immunolabeling, immunohistochemistry, immunocytochemistry, Western blotting, dot blotting, flow cytometry, fluorescent activated cell sorting (FACS), bead assays, ELISA, microarrays, capillary electrophoresis, multiplex analysis, chromatography, sensors and microfluidic systems.

Such analytical methods may also include methods for detecting an antigen on a tissue or a molecular target biological sample comprising contacting said tissue or biological sample with a nanoparticle composition as described above and detecting the presence of nanoparticles bound to the tissue or biological sample by the targeting molecule by detecting a signal from the reporter molecule.

Such analytical methods may also include a method of imaging or delivering specific effects to a target biological sample, tissue, organ, animal, human comprising adding to said target a nanoparticle composition as described above and detecting the presence of nanoparticles by detecting a signal from the reporter molecule or sensitizing the nanoparticles by providing stimulation to the labels.

Such analytical methods may also include a method of detecting comprising magnetic resonance imaging, positron emission tomography, photo acoustic imaging, computed tomography, single-photon emission computed tomography, radio-sensitization, and photo-sensitization

In certain aspects, the present invention comprises a method for preparing an organic nanoparticle, comprising the steps of:
(a) preparing an organic nanoparticle having an external wall, an inner core region, and, optionally, interior wall portions defining therein additional inner core regions;
(b) attaching a targeting molecule linked to said external wall by coupling said targeting molecule to the external wall; and
(c) incorporating reporter molecules to said nanoparticle through at least two different methods selected from the group consisting of:
   (i) encapsulation of reporter molecules in one or more inner core regions;
   (ii) embedding of reporter molecules in one or more of said external wall and said interior wall portions;
   (iii) chemical linkage of reporter molecules to one or more of said external wall and said interior wall portions;
   (iv) specific binding of reporter molecules to binding partners in one or more of said external wall and said interior wall portions; and
   (v) electrostatic binding of reporter molecules to one or more of said external wall and said interior wall portions.

Step (a) above may comprise preparing either a lipo some, a micelle, a multilamellar vesicle, or a multi-vesicular vesicle.

In such preparatory methods, the targeting molecule may in certain embodiments be an antibody, antibody fragment, peptide, nucleotide, aptamer, biotin, avidin, streptavidin, NeutrAvidin, CaptAvidin, or folic acid linked to said external wall. In certain other methods, the reporter molecules are selected from the group consisting of a dye, a chelation complex, a coordination complex, an enzyme, an enzymatic substrate, a colorimetric substrate, a semiconductor nanocrystal, a mass tag, or a Raman tag.

### BRIEF DESCRIPTION OF THE DRAWINGS

FIG. 1 is a schematic drawing of a multi-loaded nanoparticle having an interior space 1 contained within a shell 2.

FIG. 2 is a schematic drawing of targeted multi-loaded nanoparticle.

FIG. 3 is a schematic drawing of the cross-section of a targeted multi-loaded nanoparticle in multi-lamellar form.

FIG. 4 is a schematic drawing of a complex multi-loaded nanoparticle with multilayer structure, such as a multi-vesicular vesicle (MVV).

FIG. 5A-B is a pair of images showing membrane marker CD20 (B-lymphocyte antigen cluster of differentiation 20) immunofluorescent staining of tonsil tissue formalin-fixed, paraffin-embedded (FFPE) sections.

FIG. 6 is a graph showing fluorescence intensity of tonsil tissue CD20 immunofluorescent staining.

### DETAILED DESCRIPTION OF THE PREFERRED EMBODIMENT

### Overview

Described herein is a nanoparticle composition, preferably lipid-based, that includes reporters or detectable labels that are associated with the nanoparticle through multiple modes of physical and/or chemical interactions. For example, reporter molecules are linked simultaneously through internal and external surface conjugation, surface adsorption, encapsulation, and embedding in the shells. The lipid based nanoparticles, when prepared by proper synthesis schemes or chemical modifications, provide targeting capability and preferentially bind to specific molecular targets, such as for example, antibodies, cell surface markers, and antigens. The lipid-based nanoparticles can be used, for example, in amplification or enhancement of signals for detection, labeling, imaging, multiplex analyses, radio-sensitization, photo-sensitization, diagnostic and analytical purposes. With targeting capacity, i.e. the inclusion of molecules that bind specifically to predetermined targets in a complex mixture, the nanoparticles are termed “targeted, multi-loaded lipid nanoparticles”.

Surface conjugation, encapsulation, and embedding are the most ready modes of association available to lipid-based nanoparticles. It has been known that each mode has certain limitations on the amount of reporters that can be loaded, mainly due to the requirements for structure integrity, stability, and reporter function. For example, the overall fluorescence intensities of carboxyfluorescein and sulforhodamine, two commonly used fluorophores in biological imaging, do not have a linear relationship with the concentrations of fluorophores encapsulated in liposomes. The signals from carboxyfluorescein and sulforhodamine have been found to peak at approx. 10 mM and approx. 1 mM, respectively.⁸ Higher loading of these dyes result in “overcrowding” of dye molecules and self-quenching, leading to inverse relationship between the resulting fluorescence intensity and the fluorophore concentration. For another example, loading of 4-Di-16-ASP4-(4-(dihexadecylamino)styryl)-N-methylpyridinium iodide (“DiA”), one of the commonly used fluorescent membrane tracers and carries a +1 charge, via lipid bilayer embedding over approximately 10 mol % in buffered saline leads to precipitation, likely due to overcharging of the lipid nanoparticle. Lastly, surface conjugation requires suitable functional groups present in both the lipid bilayers and the reporters, thus potentially leading to destabilization of the lipid bilayers, integrity of liposomes, or loss of function for the reporters due to chemical modification.

Therefore, increasing the loading of reporters via simultaneous engagement/activation of multiple modes of association becomes a route to further enhance reporter signals. For fluorescence- and colorimetric-based detections, the reporters associated via different modes are typically required to present the same spectral properties, or at least similar enough so that the overall properties satisfy the intended applications. On the other hand, the associated reporters can have different spectral properties that are designed so that reporters facilitate the function of each other; for example, reporter 1 facilitates the function of reporter 2, which facilitates the function of reporter 3, etc. Such nanoparticles are suitable for applications such as FRET (fluorescence resonance energy transfer). For metal-based detection, the reporters (mass tags) that are associated via different modes can be present in different chemical forms or environment, such as chelators, conjugated chelators, functionalized chelators, and multiple types of chelators. Metal-based detection mainly relies on the mass signatures of the elements; therefore, the atomic mass and the isotopic number are the main considerations.

A liposome that is loaded with both encapsulated and surface-immobilized fluorophores has been reported.⁹ A liposome that is loaded with both encapsulated and embedded metal chelators has been reported.¹⁰ However, no targeting function was implemented, which requires additional design considerations and synthesis techniques. This application covers the design of lipid nanoparticle compositions that carry reporters associated via a defined number of chemical/physical interactions with targeting capability, that the nanoparticles can be used for detection, labeling, imaging, diagnostic and analytical purposes. Exemplary applications include immunohistochemistry, immunocytochemistry, flow cytometry, fluorescent in situ hybridization (FISH), microarrays, enzyme-linked immunosorbent assays (ELISA), western blotting, dot blotting, capillary electrophoresis, bead assays, multiplex assays, mass cytometry, secondary ion mass spectrometry, metal-sensitive detection and imaging, rapid diagnostic tests, radio-sensitization, photo-sensitization, ex vivo imaging, in vivo imaging, and so on.

### DEFINITIONS

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

The term “binding partner” means, with respect to a given molecule such as a reporter molecule, a counterpart molecule that binds specifically to the given molecule through a molecular recognition between the two. Examples are biotin binding to members of the avadin family.

The term “vesicle”, as known in the art and used herein, refers to a small enclosed structure. Often the structures are membranes composed of lipids, polymers other structural components associated with membranes. The vesicles are synthetic and contain a generally continuous outer wall.

The term “nanoparticle” is used herein to refer to an organic particle typically having a diameter 20 to 1,000 nm, or in some embodiments 300 nm or less and composed of an assembly of individual polymeric molecules that are covalently or noncovalently bound in an ordered fashion. The main example of a nanoparticle as used herein is a liposome or micelle. The term “liposome”, as is known in the art, refers to vesicles surrounded by a bilayer formed of components usually including lipids optionally in combination with non-lipidic components. They may be unilamellar or multilamellar.

The term “organic nanoparticle” is used herein to refer to a nanoparticle formed from polymeric organic materials, such as lipids or polyesters, etc., as opposed to nanoparticles which are completely inorganic, e.g. gold. An example of an organic nanoparticle is a liposome, which, as is known in the art, refers to vesicle comprised of one or more concentrically ordered lipid bilayers encapsulating an aqueous phase. Liposomes can carry inorganic components, such as metal elements and chelates. Formation of such vesicles requires the presence of “vesicle-forming lipids” which are amphipathic lipids, such as phosphatidylcholine, capable of either forming or being incorporated into a bilayer structure. The latter term includes lipids that are capable of forming a bilayer by themselves or when in combination with another lipid or lipids. An amphipathic lipid is incorporated into a lipid bilayer by having its hydrophobic moiety in contact with the interior, hydrophobic region of the membrane bilayer and its polar head moiety oriented toward an outer, polar surface of the membrane. Hydrophilicity arises from the presence of hydrophilic head groups as well as functional groups such as hydroxyl, phospho, carboxyl, amino or sulfhydryl groups. Hydrophobicity results from the presence of a long chain of aliphatic hydrocarbon groups. Cholesterol is often included in the lipid composition even though it does not possess the traditional lipid structure described above. Depending on the composition, liposomes can exhibit a range of physical and chemical properties that can be tuned to suit intended applications. Examples of derivatized liposomes or liposomes with particular compositions include flexible liposomes, transferosomes, solid lipid nanoparticles, niosomes, cerasomes, nanoemulsions, and so on.

Nanoparticles may also include polymersomes, a class of artificial vesicles, tiny hollow spheres that enclose a solution. Polymersomes are made using amphiphilic synthetic block copolymers to form the vesicle membrane, and have radii ranging from 50 nm to 5 μm or more, described e.g. in Nam et al. U.S. Pat. No. 6,569,528, entitled “Amphiphilic biodegradable block copolymers and self-assembled polymer aggregates formed from the same in aqueous milieu” which describes amphiphilic biodegradable block copolymers comprising polyethylenimine (PEI) as a hydrophilic block and aliphatic polyesters as a hydrophobic block, which can form various size of polymer aggregates and have very low critical micelle concentration.

The term “micelle,” as used herein and known in the art, refers to an aggregate of surfactant molecules dispersed in a liquid colloid. A typical micelle in aqueous solution forms an aggregate with the hydrophilic “head” regions in contact with surrounding solvent, sequestering the hydrophobic single tail regions in the micelle center.

The term “tandem dye,” as used herein and known in the art refers to a dye such as Cy7PE, Cy7APC etc., that is made by linking two fluorochromes. As is known, Cy7 refers to cyanine 7, PE refers to phycoerythrin; and APC refers to allophycocyanin.

The term “targeting moiety” as used herein refers to a molecule attached to the nanoparticle in order to direct the nanoparticle to a particular ligand and bind to that ligand, with the nanoparticle. Targeting moieties include antibodies, antibody fragments, such as Fab portions, biotin, avidin, streptavidin, NeutrAvidin (deglycosylated native avidin), CaptAvidin (biotin binding protein), glycoprotein (e.g., transferrin), hyaluronic acid, RGD, NGR peptide, receptor ligand (e.g., vascular endothelial growth factor, VEGF), nucleotide, peptide, antagonist G, or folic acid.

The term “chelate” as used herein refers to a complex of a central atom, a substrate, or a metal element and one or more chelators, chelants, chelating agents, ligands, complexing agents, or sequestering agents joined through coordinate bonds. A chelate can be a chelate complex, or a coordination complex.

The term “antibody mimetic” as used herein refers to organic compounds that, like antibodies, can specifically bind antigens, but are not structurally related to antibodies. They are artificial peptides, proteins, nucleic acids, and small molecules that exhibit antibody-like properties, but not artificial antibodies, antibody fragments and fusion proteins composed from these. Examples are Affibody molecules, Affilins, Affitins, Anticalins, Avimers, DARPins, Fynomers, Kunitz domain peptides, and Monobodies.

### General Methods and Materials

### Nanoparticles

The exemplified liposomes used herein may be prepared by a number of known methods. Any suitable vesicle-forming lipid may be used. Examples can be seen in Szoka, Jr. and Papahadjopoulos.¹¹ This includes phospholipids such as phosphatidylcholine (PC), phosphatidylglycerol (PG), phosphatidylinositol (PI), phosphatidic acid (PA), phosphatidyethanolamine (PE) and phosphatidylserine (PS); glycolipids; and sphingolipids such as sphingosine, ceramides, sphingomyelin, glycosphingolipids (such as cerebrosides and gangliosides), and sterol lipids (such as cholesterol).

Liposomes can be generated by conventional techniques used to prepare vesicles. These techniques include the ether injection method (Deamer et al., Acad. Sci. (1978) 308: 250), the surfactant method (Brunner et al., Biochim. Biophys. Acta (1976) 455: 322), the freeze-thaw method (Pick et al., Arch. Biochim. Biophys. (1981) 212: 186) the reverse-phase evaporation method (Szoka et al., Biochim. Biophys. Acta. (1980) 601: 559 71), the ultrasonic treatment method (Huang et al., Biochemistry (1969) 8: 344), the ethanol injection method (Kremer et al., Biochemistry (1977) 16: 3932), the extrusion method (Hope et al., Biochim. Biophys. Acta (1985) 812:55 65) and the french press method (Barenholz et al., FEBS Lett. (1979) 99: 210). All of the above processes are basic technologies for the formation of liposome vesicles and these processes can be used in combinations. Preferably, small unilamellar vesicles (SUVs) are prepared by the extrusion method, the ultrasonic treatment method, the ethanol injection method and the French press method. Preferably, multilamellar vesicles (MLVs) are prepared by the reverse-phase evaporation method or by the simple addition of an aqueous solution to a lipid film followed by dispersal by mechanical agitation (Bangham et al., J. Mol. Biol. (1965) 13: 238 252).

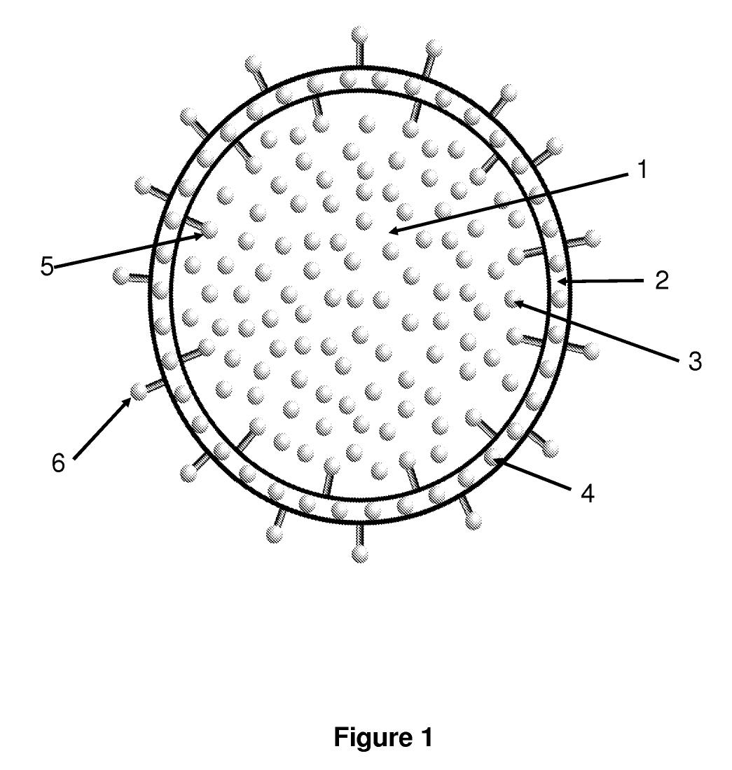

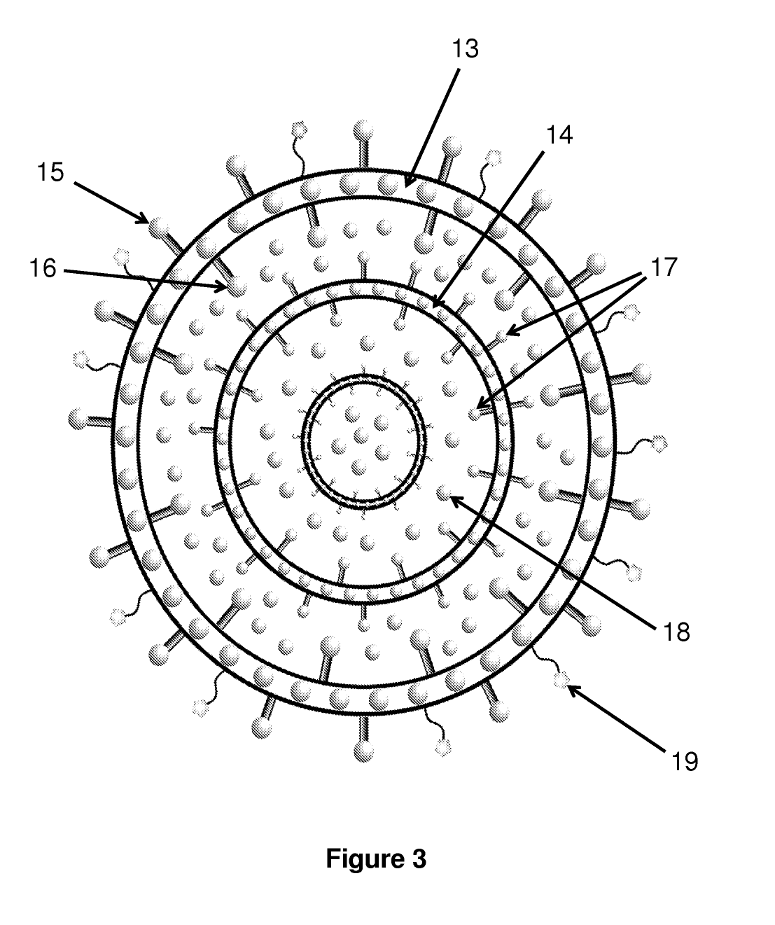

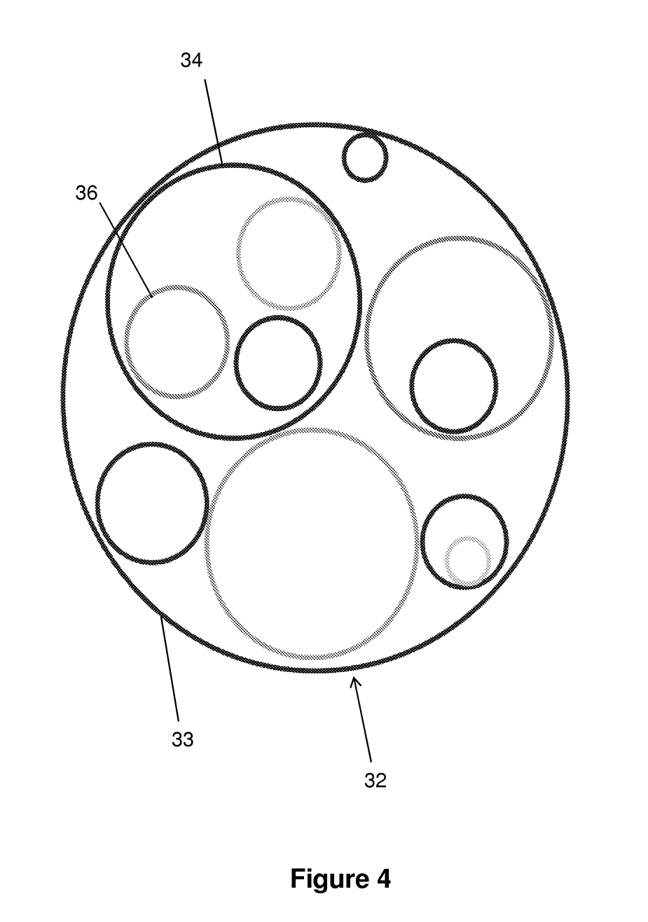

Referring to FIG. 4, multiwalled nanoparticles can be made according to a variety of methods. Preferred methods rely on the fact that, when lipid films are agitated in aqueous solution, multilamellar and complex forms of lipid assemblies form. Methods of preparation and characterization can be found in Rongen et al. J. Immunol. Meth. 1997, Vol. 204, No. 2, pp. 105-133 (FIG. 3) and Gomez-Henz and Fernandez-Romero, Trends in Analytical Chemistry, 2005, Vol. 24, No. 1, pp. 9-19 (FIG. 1), cited in the references. Various types of liposomes may be prepared; methods for preparing various types of liposomes are described in Avanti Polar Lipids' Technical Support Literature, available on the company's web site under Technical Support. Using rehydration, sonication, extrusion, agitation, organic-aqueous phase mixing, or combination of the above techniques can lead to various complex lipid structures. As described by Avanti Polar Lipids, the product of hydration is a large, multilamellar vesicle (LMV) analogous in structure to an onion, with each lipid bilayer separated by a water layer. The spacing between lipid layers is dictated by composition with poly-hydrating layers being closer together than highly charged layers which separate based on electrostatic repulsion. Once a stable, hydrated LMV suspension has been produced, the particles can be downsized by a variety of techniques, including sonication and extrusion.

Further guidance may be found in “Generation of multilamellar and unilamellar phospholipid vesicles,” M. J. Hope, M. B. Bally, L. D. Mayer, A. S. Janoff and P. R. Cullis, Chemistry and Physics of Lipids Volume 40, Issues 2-4, June-July 1986, Pages 89-107.

### Multi-Layer Nanoparticle

In FIG. 4, the nanoparticle 32 comprises an outer layer defining a closed interior space (e.g. a lipid bilayer outer membrane) that contains within it smaller nanoparticles 34 that that may also comprise outer membranes and an interior space holding a still smaller nanoparticle 36.

Also contemplated are concentric multiphasic nanoparticles, such as described e.g. in Foldvari, U.S. Pat. No. 5,853,755, entitled “Biphasic multilamellar lipid vesicles.” As described there, when using such a lipid-based nanoparticle, the vesicle has a central core compartment that contains an emulsion with oil as the dispersed phase. Water-soluble ingredients can be dissolved in the water phase of the emulsion. Oil-soluble ingredients can be dissolved in the oil droplets of the emulsion, or can be emulsified. The central core compartment is surrounded by a multitude, at least about fifteen and normally many more, substantially concentric spherical lipid bilayers. Between each adjacent pair of lipid bilayers is a space that is occupied by water or aqueous solution. In that water there can again be emulsified oil droplets or emulsified lipophilic substances or both.

Water-soluble substances can be dissolved in the water and lipophilic substances can be dissolved in emulsified oil droplets or be emulsified as dispersed phase in the water. Lipophilic ingredients can be incorporated actually within the lipid bilayer. It is also contemplated that vesicles, e.g. micelles, may be associated on the outside of the outer layer.

### Reporter Molecules

Reporter molecules may be dyes (discussed below), fluorophores, proteins, such as enzymes (e.g. horseradish peroxidase, alkaline phosphatase, beta galactosidase) or phycobiloprotein, or enzymatic substrates, or mass tags (e.g., lanthanides), or chelates, or mass tags, or Raman tags, or radio-sensitizers, or photo-sensitizers, or semiconductor nanocrystals. They are increasingly used in biomedical applications, because they provide strong and signature-like signals.

Phycobiliproteins are water-soluble proteins present in cyanobacteria and certain algae (rhodophytes, cryptomonads, glaucocystophytes) that capture light energy, which is then passed on to chlorophylls during photosynthesis. Phycobiliproteins are formed of a complex between proteins and covalently bound phycobilins that act as chromophores (the light-capturing part). They are most important constituents of the phycobilisomes.

Enzymes, substrates and other molecular entities that provide or can be converted into identifiable signals, or signatures, can be incorporated into lipid nanoparticles such as micelles and liposomes by various methods. These methods are often combinations of specific compositions, functional groups, synthesis schemes, conjugation reactions, and formulation techniques, and are termed “modes” in the following description. These modes represent different ways and physical/chemical interactions the reporter molecules can associate with the nanoparticle. For example, liposomes can carry multiple reporters in their interior space, in the lipid layers, and on the internal and/or external surfaces of the lipid layers; micelles can carry multiple reporters in between their lipid tails or on their lipid heads.

For a reporter molecule to associate with a liposome via encapsulation, generally the reporter has to be soluble and remains stable in the aqueous phase in which the liposome is formulated. When the liposomes are assembled, the reporters are confined in the lipid sheet enclosure. For a reporter to associate with a lipid nanoparticle via lipid layer embedding, generally the reporter has to carry a lipophilic portion that presents adequate affinity with other lipids in the lipid layers. When the lipids self-assemble into lamellar structures, the reporters are organized within the lipid layers. For a reporter to associate with a liposome via surface conjugation or adsorption, the reporter has to carry proper chemical groups that can be chemically or physically linked with the corresponding chemical motifs on the internal or external surface of the lipid layers. These are among the typical modes of association for lipid nanoparticles.

FIG. 1 shows a schematic of the different types of reporter molecules located within the multi-loaded nanoparticle. The interior space 1 contains encapsulated reporter molecules 3 dispersed within the interior region. The shell 2 comprises a bilayer, such as a liposome, having between the layers embedded reporter molecules 4. On the shell are reporter molecules 5 associated on the inner surface, and reporter molecules 6 associated on the outer surface.

One preferred reporter molecule is a fluorescent dye. Useful fluorescent dyes in the present compositions include but are not limited to, fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g. Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), HiLyte Fluor™ dyes, eFluor dyes, and the like. For more examples of suitable fluorescent dyes and methods for coupling fluorescent dyes to other chemical entities see, for example, “The Handbook of Fluorescent Probes and Research Products”, 9.sup.th Ed., Molecular Probes, Inc., Eugene, Oreg. Also suitable is Cascade Blue dye, available from Life technologies. Cascade Blue fluorophore shows less spectral overlap with fluorescein, an important advantage for multicolor applications. In addition, this reactive Cascade Blue derivative has high absorptivity, is highly fluorescent and, unlike most dyes, resists quenching upon protein conjugation.

Another type of reporter molecule that may be used with the present invention is a metal chelate such as an iron chelate, a copper chelate, a cobalt chelate, a lanthanide chelate. Lanthanide chelates and methods for incorporating a lanthanide chelate into a nanoparticle are described in US 20070286810A1, “NANOPARTICLES COMPRISING LANTHANIDE CHELATES.” A number of lanthanide (e.g. terbium, europium) complexes are known and additional lanthanide complexes useful here are described in U.S. Pat. No. 6,740,756, “Fluorescent lanthanide chelates.” Useful lanthanides in the present compositions include but are not limited to, Dy164, Er166, Er167, Er168, Er170, Eu151, Eu153, Gd156, Gd158, Gd160, Ho165, La139, Nd142, Nd144, Nd145, Nd146, Nd148, Nd150, Lu174, Lu175, Pr141, Sm147, Sm152, Sm154, Tb159, Tm169, Yb171, Yb172, Yb174, Yb176.

### Multiple Dyes

In certain embodiments, the present invention comprises the use of multiple dyes and multiple modes of association, where the dyes are chosen so as to interact based on the concentration and locations of the dyes within the nanoparticle. The present nanoparticles may be packed with a dye at a concentration in which the dye molecules interfere with each other until the nanoparticle is disrupted, “unpacking” the dyes and increasing fluorescence. Conversely, the nanoparticle may be loaded with dye molecules at a high concentration, but, because the dye molecules are associated in different ways in different parts of the nanoparticles, the molecules do not interfere with each other, allowing for the preparation of a brighter composition.

Dyes used herein may be selected on principles known in choosing dye pairs for FRET. Fluorescence resonance energy transfer or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly.

Based on principles of FRET, one may employ tandem dyes, i.e. two different dyes that produce a signal based on their proximity-based interaction. Tandem dyes exploit the principle of FRET. A donor chromophore (e.g., phycoerythrin [PE], allophycocyanin [APC]) is excited by a suitable light source and transfers its absorbed energy to an acceptor fluorophore, which then emits this energy as fluorescence at the acceptor-dye wavelength. Because the energy-transfer efficiency depends on the distance between the donor and acceptor chromophores, it is important to conjugate or position them in close proximity. One approach to this process uses a method of intramolecular unfolding of the donor phycobiliprotein molecules (e.g., PE or APC). This makes it possible for the acceptor-reactive-dye molecules to react preferentially with the newly exposed hydrophobic regions containing the bilin chromophores. For example, the tandem dye Cy5PE can be excited at donor excitation wavelengths but emit at acceptor emission wavelengths. In the case of Cy5PE, this means the molecule can be excited at 488 nm (a wavelength which does not excite Cy5 at all) and emit at 680 nm (at which PE emits extremely little light). In essence, the tandem dyes shift the emission spectrum to lower wavelengths.

### Multiple Metals

In certain embodiments, the present invention comprises the use of multiple metals and multiple modes of association, where the metals are chosen so as to interact based on the concentration and locations of the metals within the nanoparticle. Metals can be dissolved or incorporated in the ion form, in the salt form, or more commonly, in the chelated form. Common chelators include ethylenediaminetetraacetic acid (EDTA), pentetic acid or diethylene triamine pentaacetic acid (DTPA), 1,4,7,10-tetraazacyclododecane-1,4,7,10-tetraacetic acid (DOTA), and so on that, according to American Society for Testing and Materials (ASTM)-A-380, are “chemicals that form soluble, complex molecules with certain metal ions, inactivating the ions so that they cannot normally react with other elements or ions to produce precipitates or scale.” In one embodiment in which gadolinium is associated through both encapsulation and surface conjugation, gadobenate dimeglumine (Gd-BOPTA, Multihance®) is encapsulated in the interior of the nanoparticle, and 1,2-dimyristoyl-sn-glycero-3-phosphoethanolamine-N-diethylenetriaminepentaacetic acid gadolinium salt (14:0 PE-DTPA(Gd)) is incorporated in the lipid layers of the nanoparticle. Metal elements can be associated with the nanoparticle through different types of chelators described above, and different metal elements can be incorporated.

### Different Linkages of Reporter Molecules

To provide a stable lipid nano-construct with multiple association modes available, the liposome has certain compositional and functional requirements. In one embodiment, the composition of liposome, before additional reporters and targeting moieties are incorporated, is 20-80 mol % 1,2-distearoyl-sn-glycero-3-phosphocholine (DSPC), 10-60 mol % cholesterol, 0.1-20 mol % 1,2-distearoyl-sn-glycero-3-phosphoethanolamine-N-[methoxy(polyethylene glycol)-2000] (mPEG₂₀₀₀-DSPE), 0.1-10 mol % 1,1′-dioctadecyl-3,3,3′,3′-tetramethylindocarbocyanine perchlorate (DiIC18(3)), 0.1-10 mol % 1,2-distearoyl-sn-glycero-3-phosphoethanolamine-N-[amino(polyethylene glycol)2000] (amine-PEG₂₀₀₀-DSPE), and 0.1-10 mol % 1,2-distearoyl-sn-glycero-3-phosphoethanolamine-N—[maleimide(polyethylene glycol)2000] (maleimide-PEG₂₀₀₀-DSPE), but not limited to such. In the composition, DSPC and cholesterol mainly provide the structural integrity; mPEG₂₀₀₀-DSPE provides the PEGylated coating on the surface that prevents non-specific adhesion or binding of with samples in analysis; DiIC18(3) is the fluorescent reporter exhibiting excitation maximum near 549 nm and emission maximum near 565 nm, amine-PEG₂₀₀₀-DSPE provides primary amine groups for conjugation with additional reporters or targeting moieties that carry functional groups reactive towards primary amines; maleimide-PEG₂₀₀₀-DSPE provides maleimide groups for conjugation with additional reporters or targeting moieties that carry functional groups reactive towards maleimide. Poly(ethylene glycol) (PEG) spacer is often included in nanoparticles or modified on biomacromolecules such as proteins to reduce non-specific binding.^{12,13} PEG can be incorporated into liposomes at, for example, 0.01-30 mol % on the basis of total lipids.¹³

It is known in the art that fluorescent molecules quench one another when they are in proximity, e.g., a few nanometers in distance. This phenomenon called fluorescence quenching (http (colon slash slash) en.wikipedia.org/wiki/Fluorescence_quenching) prohibits unlimited increase in fluorescence intensity by simply increasing the concentration of fluorophores loaded into a lipid nanoparticle. However, fluorescence quenching can be intentionally exploited in the process of, for example, fluorescence resonance energy transfer (FRET) mechanism that is commonly used in biological and biomedical assays.

The definition and detailed description of these modes of association are described as follows.

### Association of Reporters Through Encapsulation

Encapsulation is a mode of association in which the reporter molecules are present in the enclosure or enclosed compartments of the nanoparticle, typically without direct physical or chemical interactions with the nanoparticle constituents. The reporters can be soluble, crystallized, or stabilized in the medium, or recipients, or facilitators within the enclosure or enclosed compartments. For example, hydrophilic dyes such as carboxyfluorescein and sulforhodamine are soluble in aqueous medium, e.g., physiological buffers, and can be encapsulated in liposomes. For example, metal chelates such as diethylene triamine pentaacetic acid gadolinium coordination complex (DTPA(Gd)), or complexes of DTPA and other metals, can be encapsulated in liposomes.

Liposomes and micelles can be formed by homogenizing lipid films that are hydrated in aqueous suspension. During the hydration and homogenizing process, lipid films may enclose to form spherical nanoparticles and segregate the interior and exterior spaces of the nanoparticle. Reporters that are encapsulated in the interior are thus protected and form a single entity with the liposome. Reporters can be encapsulated by either active loading or passive loading. Active loading requires that the chemical structure and properties of the reporters resemble the behavior of, for example, doxorubicin. With chemical structure and properties similar to doxorubicin, the reporter can be actively loaded into the interior of liposomes against concentration gradient across the lipid membranes, thus achieving an extraordinary high concentration and extremely efficient loading. On the other hand, passive loading is less demanding in the chemical properties of the reporters. In general, the reporters are dissolved and form a homogeneous phase in the suspension in which liposomes are prepared.

### Association of Reporters Through Conjugation

Conjugation involves covalent bonding of two chemical entities. Dyes and fluorophores with functional groups available for conjugation can be linked to one of the external or interior surfaces of the nanoparticles by covalent coupling. Lipid bilayers can be prepared with functionalized lipids for coupling to these dyes and fluorophores. The same scheme can be used for metal chelators. Many chelators are modified with functional groups available for covalent coupling with lipids. Examples of chelator-lipid conjugates are 1,2-distearoyl-sn-glycero-3-phosphoethanolamine-N-diethylenetriaminepentaacetic acid and 1,2-dioleoyl-sn-glycero-3[(N-(5-amino-1-carboxypentyl)iminodiacetic acid)succinyl]. Such lipids are commercially available, e.g. from Avanti Polar lipids, or their synthesis and modification schemes are known in the art. The conjugation reactions can involve a crosslinker to activate the functional groups from either or both entities. A bifunctional crosslinker useful to the composition comprises two different reactive groups capable of coupling to two different functional entities. The two reactive groups can be the same or different and include, but are not limited to, reactive groups such as thiol, carboxylate, carbonyl, amine, hydroxyl, aldehyde, ketone, active hydrogen, ester, sulfhydryl or photoreactive moieties. A crosslinker can have one amine-reactive group and a thiol-reactive group on the functional terminals. Further examples of heterobifunctional cross-linkers that may be used as linking agents in the invention include, but are not limited to:

amine reactive+sulfhydryl-reactive crosslinkers

amine reactive+carbonyl-reactive crosslinkers

carbonyl-reactive+sulfhydryl-reactive crosslinkers

amine-reactive+photoreactive crosslinkers

sulfhydryl-reactive+photoreactive crosslinkers

carbonyl-reactive+photoreactive crosslinkers

carboxylate-reactive+photoreactive crosslinkers

arginine-reactive+photoreactive crosslinkers

Below is a list of categories in which crosslinkers generally fit. The list is exemplary and should not be considered exhaustive of the types of crosslinkers that may be useful for the invention. For each category, i.e. which functional group these chemicals target, there are some subcategories, because one reactive group is capable of reacting with several functional groups.

Most crosslinkers with reactive groups can be broadly classified in the following categories:

TABLE 1
Crosslinkers

| | |
|---|---|
| Amine-reactive | the cross-linker couples to a amine (NH₂) |
| | containing molecule |
| Thiol-reactive | the cross-linker couples to a sulfhydryl (SH) |
| | containing molecule |
| Carboxylate-reactive | the cross-linker couples to a carboxylic acid |
| | (COOH) containing molecule |
| Hydroxyl-reactive | the cross-linker couples to a hydroxyls (—OH) |
| | containing molecule |
| Aldehyde- and ketone- | the cross-linker couples to an aldehyde |
| reactive | (—CHO) or ketone (R₂CO) containing |
| | molecule |
| Active-hydrogen-reactive | the cross-linker couples to an |
| | active-hydrogen-containing compound |
| Photo-reactive | the cross-linker is activated by irradiation of |
| | certain energy |
| Carbohydrate-reactive | the cross-linker couples to a carbohydrate |
| | containing molecule |

More specifically, chemicals entering in these categories include, but are not limited to those containing:

TABLE 2
Functional Groups

| | |
|---|---|
| 1 | Isothiocyanates, isocyanates, acyl azides, NHS esters, sulfonyl |
| | chlorides, aldehydes and glyoxals, epoxides and oxiranes, |
| | carbonates, arylating agents, imidoesters, carbodiimides, |
| | anhydrides, alkynes |
| 2 | Haloacetyl and alkyl halide derivates, maleimides, aziridines, acryloyl |
| | derivatives, arylating agents, thiol-disulfides exchange reagents |
| 3 | Diazoalkanes and diazoacetyl compounds, such as |
| | carbonyldiimidazoles and carbodiimides |
| 4 | Epoxides and oxiranes, carbonyldiimidazole, oxidation with |
| | periodate, N,N′-disuccinimidyl carbonate or N-hydroxylsuccimidyl |
| | chloroformate, enzymatic oxidation, alkyl halogens, isocyanates |
| 5 | Hydrazine derivatives for Schiff base formation or reduction |
| | amination |
| 6 | Diazonium derivatives for Mannich condensation and iodination |
| | reactions |
| 7 | Aryl azides and halogenated aryl azides, benzophenones, diazo |
| | compounds, diazirine derivatives |

In some embodiments, association of dyes and fluorophores with one of the modes in the nanoparticles can be accomplished without a crosslinker; for example, dye and fluorophores already derivatized with active chemical groups for specific conjugation reactions partially listed in the table above.

For each of these categories and subcategories there are many examples of chemicals. All these chemicals and the above list of subcategories are described in the prior art, but many can be found in, “Bioconjugate Techniques” by Greg T Hermanson, Academic Press, San Diego, 2008, which is hereby incorporated by reference.¹⁴

Association of Reporters Through Embedding into a Bilayer Wall

The shell of a lipid nanoparticle typically consists of lipid sheets, e.g., lipid bilayers. Many lipophilic dyes tend to associate with lipid bilayers and form stable structures without compromising the integrity of the lipid bilayers. The portion of the lipophilic dyes that exhibit the optical or spectral properties to be harvested in practical applications can physically be at the end, or in the middle of, or extruding from the surface of the lipid sheets. Such reporters can be incorporated by mixing the reporters in the lipid films during preparation of the lipid films. Many such reporters are described below. The physical and chemical characteristics of the embedded reporters may alter the properties of liposomes; for example, density, charge, zeta potential, transition temperature, etc. Therefore, selection of the reporters and their quantity in terms of composition percentage are among the considerations when designing and synthesizing the nanoparticle.

### Association of Reporters Through Specific Binding

Specific binding such as enzyme-substrate and ligand-receptor interactions can be used as the mode of linkage between the reporter molecules and the nanoparticle. For example, biotin and its binding partners, e.g., avidin, streptavidin, NeutrAvidin, CaptAvidin, etc., form a strong and stable complex when they interact. It is similar to enzymatic reaction but no modification of the chemical structures takes place. The binding partners are not covalently bonded, yet the association is extremely stable, with a dissociation constant of about 1.3×10⁻¹⁵ M. The reporter molecule can be modified with biotin, or biotinylated, and couple to streptavidin molecules immobilized on the nanoparticle surfaces via chemical bonding. Or, the reporter molecules can be linked to strepavidin, and coupled to the biotinylated surfaces of the nanoparticle. Liposomes, micelles, and other lipid-based nanoparticles can be prepared so that the composition contains biotinylated lipids, or lipids modified with avidin, streptavidin, or NeutrAvidin. In such cases, liposomes become targeted as the binding will only occur between specific partners.

### Association of Reporters Through Electrostatic Interactions

Electrostatic interactions refer to the physical attraction or repulsion due to Coulomb force. By preparing the liposomes with proper constituents, for example, with certain percentage of positively charged lipids, positively charged surface on the outer and inner wall of the nanoparticle is formed. Negatively charged molecules are expected to attach on the surface more to certain extent and favorably compared to neutral (non-charged) molecules. Or, the electrostatic interaction can occur between two ionic molecules, one present in the lipid nanoparticle and the other separate but attached. For example, an ionic or an ionizable head group-containing lipid can be incorporated in the lipid bilayers and associated with an ionic reporter

### Signal Amplification Via De-Quenching of Reporters

Self-quenching of fluorophores can be exploited in some applications to further enhance the overall signals, improve the sensitivity, or reduce the background. For example, when liposomes loaded with high concentrations of fluorophores, often via encapsulation, that results in self-quenching, the fluorophores can be released once the liposomes have accomplished intended binding or immobilization with the analytes or in the analytical processes. Liposomes can be lysed with detergents such as Triton X-100 or n-octyl-b-D-glucopyranoside, or with proteins such as complement or mellitin.⁵ Once the fluorophores are released, they are no longer in proximity and exhibit their fluorescent state with proper excitation. Dyes loaded in liposomes can be released to increase their overall fluorescence intensity if higher than optimal concentration is loaded.

### Targeting Capabilities

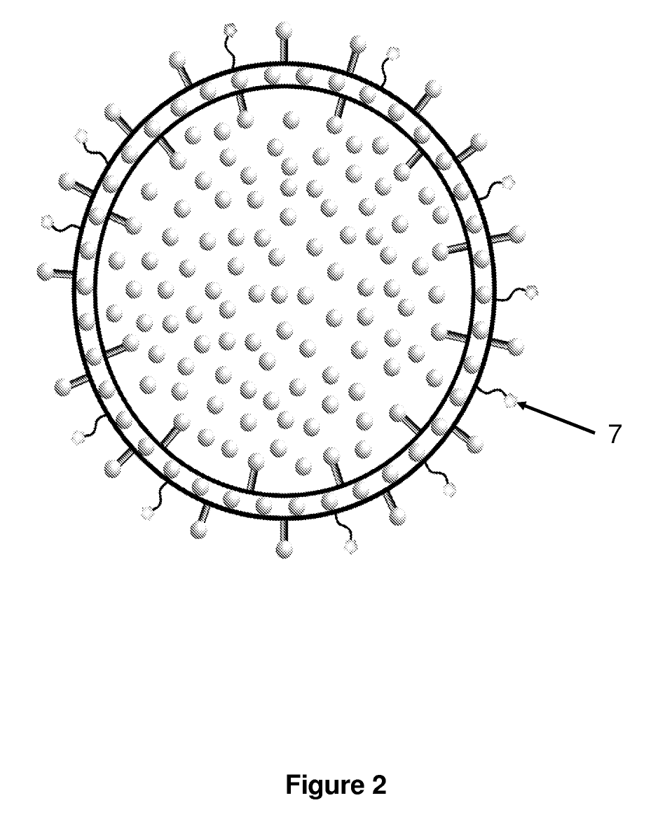

Lipid nanoparticle targeting to specific molecular entities can be achieved by associating the affinity ligands on the surface of the nanoparticles. For example, antibodies, antibody fragments, biotin, folic acids, avidin and analogs, antigen, proteins, peptides, nucleotides, oligonucleotides, sugars, and so on can be installed. As seen in FIG. 2, on the surface of the multi-loaded nanoparticle (detailed in FIG. 1), a targeting moiety 7 such as antibody, antibody fragment, biotin, etc. is linked to the shell on the outer surface.

FIG. 3 shows that the targeting moiety 19 is attached to the external surface of the nanoparticle. As can be seen, the nanoparticle has an onion-like structure, with external 13 and internal 14 layers (“walls”) of the constituent molecules (e.g., lipid) that compartmentalize the interior space. Reporter molecules 15 can be linked to the external surface outside the nanoparticles. They can also be linked to the internal surface of the external wall 16. They can also be linked to either or both sides of the internal walls that form the enclosures in the interior space 17. The reporter molecules 18 can also be encapsulated in the compartments of the nanoparticles, in between external and internal walls, without direct chemical linkage to the surface of the walls.

In general, the targeted lipid nanoparticles thus prepared are often referred to as being ligand-targeted. Many ligands are described in the Noble 2002 paper and here incorporated entirely as reference.⁴ The multi-loaded lipid nanoparticle thus bind preferentially to specific or some molecular targets, based on the affinity moiety incorporated on their surfaces.

Antibodies include polyclonal antibodies, monoclonal antibodies, synthetic antibodies, or immunogenically active fragments, or derivatives, thereof. Exemplary fragments are F(ab′)2, Fab′, Fab, scFv, heavy+light chain, and the like. Derivative include pegylated and other modified antibodies. Antibodies and fragments may be chimeric, humanized, humaneered, single-chain, or otherwise modified to modulate their affinity and/or avidity for a cellular target, immunogenicity in an organism, half life, or other physical properties.

Where the biological target is a nucleic acid, the targeting molecules may be nucleic acid probes, including DNA, RNA, and nucleic acids including synthetic bases, thiodiester bonds, end-capping groups, and other modifications.

Targeting molecules also include receptors, ligands, peptide or small-molecule binding partners, substrates, and/or inhibitors for preselected receptors, proteases, kinases, phosphatases, polymerases, growth factors, cell cycle proteins, enzymes involved in energy metabolism, structural proteins, proteins involved in mitosis or cytokinesis, and the like. An exemplary small-molecule targeting compound is folate, which targets the folate receptor.

The lipid nanoparticle typically includes one or more targeting molecules that binds specifically to a biological target.

### Methods of Use

The present nanoparticles may be used in a variety of applications. These include Western blotting, where bands of proteins are to be detected by antibodies, which here would be the targeting agent, fluorescent activated cell sorting (FACS), where the present compositions would be used for labeling and classifying the cells, ELISA, or enzyme-linked immunonosorbant assays, where the present nanoparticles would be used to either detect the antigen (direct ELISA) or detect the primary antibody (indirect ELISA). A number of immunoassays, where the signals from the bound or localized nanoparticles are detected, can be developed using the present nanoparticles. The signals can be analyzed to interpret the abundance and location of the analytes, capillary electrophoresis, where the targeted nanoparticles can be used as coating material or carrier or detecting agents for targets dispersed in the capillary tubes, sensors, where the reporters molecules serve as the indicator when the nanoparticles undergo certain interaction with the environment, microfluidic systems, where the nanoparticles provide the tracing elements for the flow and dynamics of the molecular interactions in the system.

The present nanoparticles may also be used ex vivo or in vivo for tissue imaging, or as a histochemical reagent. When used for in vivo imaging, the nanoparticle may take advantage of the enhanced permeability and retention (EPR) effect¹⁵ and achieved increased accumulation at the tumor or inflammatory sites after systemic administration. Compared to small molecule agents, biodistribution of nanoparticles is often more favorable for the purpose of imaging and/or drug delivery. Coupled with the abundant reporter molecules carried by the nanoparticles, strong signals are obtained and can facilitate the diagnosis, or image-guided operation, or study of pharmacology of the nanoparticle agents. An example can be seen in Weng et al. 2008 in which luminescent quantum dots carried by immunoliposomes accumulated extensively at subcutaneous tumors and provided in vivo fluorescence images of the tumor.¹⁶

When used as a histochemical reagent, the nanoparticles may amplify the signals from binding and provide better sensitivity, dynamic range, and contrast for the markers being analyzed. Traditionally, amplification of signals in histochemical analysis is achieved by crosslinking or polymerizing the signaling molecules through multiple reagents in multiple steps, such as the avidin-biotin systems. The nanoparticles may achieve signal enhancement without engaging additional reagents and thus simplify the procedures. The nanoparticles, by localizing an abundance of elements, may also enable metal-based detection and imagining, such as secondary ion mass spectroscopy (SIMS).

### EXAMPLES

### Example 1

### Synthesis and Use

Methods for liposome preparation are known in the art. The invention constitutes adding new components to liposomal formulations, particularly, liposomes and targeted liposomes (immunoliposomes, biotinylated liposomes, streptavidin-conjugated liposomes, etc.) loaded with fluorophores, mass tags, Raman tags, colorimetric substrates, and so on, through multiple modes of association. The following is a sample protocol for the preparation of a targeted multi-loaded lipid-based nanoparticle:

(1.) Lipid Vesicle with Probes Preparation
a. Exemplary lipid composition:

| | | Molar Percentage |
|---|---|---|
| Component | Abbreviation | in Composition |
| 1,2-distearoyl-sn-glycero-3- | DSPC | 58 |
| phosphocholine | | |
| Cholesterol | Chol | 30.5 |
| 1,2-distearoyl-sn-glycero-3- | mPEG₂₀₀₀- | 5 |
| phosphoethanolamine-N- | DSPE | |
| [methoxy(polyethylene glycol)- | | |
| 2000] | | |
| 1,1′-dioctadecyl-3,3,3′,3′- | DiIC₁₈(3)-DS | 3 |
| tetramethylindocarbocyanine-5,5′- | | |
| disulfonic acid | | |
| 1,2-distearoyl-sn-glycero-3- | amine- | 3 |
| phosphoethanolamine-N- | PEG₂₀₀₀-DSPE | |
| [amino(polyethylene glycol)2000] | | |
| 1,2-distearoyl-sn-glycero-3- | maleimide- | 0.5 |
| phosphoethanolamine-N- | PEG₂₀₀₀-DSPE | |
| [maleimide(polyethylene | | |
| glycol)2000] | | |

Note:
DiIC₁₈(3)-DS serves the embedded reporters in which the fluorescent carbocyanine is covalently linked to the lipid portion that is embedded in the lipid layers.

b. Exemplary lipid concentration: 30 mg/ml
c. Add ˜10 ml chloroform and ˜1 ml methanol
d. Vortex to mix
e. Use a rotary evaporator to remove solvent
f. Vacuum for ˜1 hr
g. Freeze-dry ˜460 mTorr ˜−56° C. for 1 hr 30 min
h. Buffer: Freshly prepared 5 mM HEPES or 5 mM phosphate, 10 mM Cy3 NHS ester, 10 mM water soluble Cy3, 135 mM NaCl, pH 8 Note: Cy3-NHS serves the conjugated reporters on the internal and external surfaces of the liposomes. Water soluble Cy3 dissolves in the buffer and serves the encapsulated reporters in the aqueous interior of liposomes.
i. Add 10 ml of buffer to the dried lipid film
j. Thaw at 60° C. and freeze on dry ice. Freeze-thaw multiple times.
k. Extrude through a 80 nm polycarbonate membrane at ˜60° C. 11 times by an extruder
l. Store at 4° C.

### (2.) Reduction of Antibody

a. Prepare 50 mM dithiothreitol (DTT) reducing agent to produce reactive groups in antibodies.
b. Note: Other useful agents for reducing antibodies include tris(2-carboxyethyl) phosphine (TCEP), mercaptoethylamine (MEA), cystamine, 2-mercaptoethanol (BME), and so on. The choice of reducing agents depends on the chemical properties of antibodies, the desired fragments of antibodies, the scheme used for fragmentation, and the ease of purification after reactions.
c. Exemplary antibody: Goat anti-mouse IgG (GAM)
d. Reduce GAM by adding 100 ul 50 mM DTT to 400 ul GAM and incubate at 37° C. for 30 min
e. Transfer to ice water bath and desalt by spin columns
f. Store at 4° C.

### (3.) Preparation of Targeted Multi-Loaded Liposomes

a. Add ˜400 ul reduced antibody to ˜575 ul lipid vesicles containing surface conjugated reporters (Cy3-NHS), encapsulated reporters (Cy3), and embedded reporters (DiIC₁₈(3)-DS).

b. Vortex

c. Incubate at RT for several hours

d. Store at 4° C.

The above steps are carried out in order in this example, and illustrate a protocol for attaching a modified antibody to a lipid whereby the antibody is displayed on the outer surface of the nanoparticle and can be present in sufficient concentration (numbers of antibodies) to cause effective targeting of the nanoparticle, in this case to mouse IgG. Another protocol for linking a protein to a liposome outer surface is described in Harokopakis et al., “Conjugation of cholera toxin or its B subunit to liposomes for targeted delivery of antigens,” J Immunol Methods. 1995 Sep. 11; 185(1):31-42. Briefly, a portion of the DPPE constituent of the liposomes was modified by using the heterobifunctional reagent SMCC. The reaction product, DPPE-MCC, was purified and used along with DSPC, POPC, and cholesterol to form a lipid film on the walls of a round-bottom glass flask. The film was hydrated with HEPES containing 265 μg of purified SBR per ml, which resulted in large multilamellar liposomes. Subsequently, small unilamellar liposomes were produced by extruding the large multilamellar liposomes through a 400-nm-pore-size, followed by a 100-nm-pore-size, membrane.

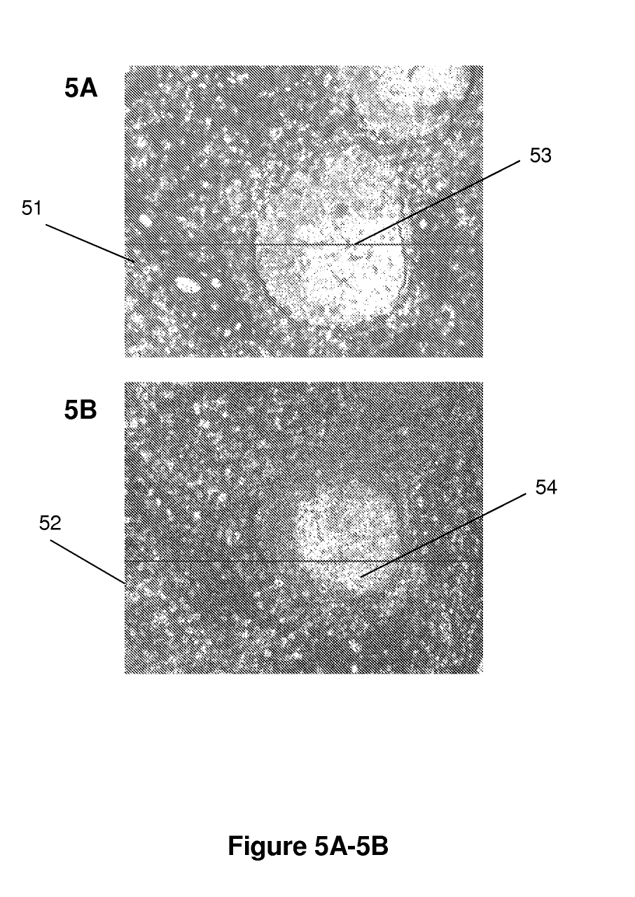

FIG. 5A-B shows images of cells stained with multi loaded nanoparticles prepared with targeting to CD20. CD20 is the molecular target of therapeutic monoclonal antibodies (mAb) rituximab, Ibritumomab tiuxetan, and tositumomab, which are all active agents in the treatment of all B cell lymphomas and leukemias.

FIG. 5A (51) shows the fluorescence microscope image of CD20 staining by targeted, multi-loaded nanoparticles (in the particular example, loaded with Cy3 equivalent dyes, prepared by the methods described in the present EXAMPLE 1. FIG. 5B (52) shows the adjacent section from the same tissue (bearing the same tissue matrix, cellular features, and biomarker expressions) stained by traditional antibody conjugate (Cy3 goat anti-mouse IgG (H+ L), Invitrogen Catalog Number A10521). Methods for slide processing and staining can be found in published protocols. Reagents and protocols have been generally optimized. Images (51 and 52) were acquired by a 40× objective and the same microscope settings with equal camera gain and offset. 53 and 54 are the lines in image 51 and 52, respectively, for which the signal intensity profiles were analyzed.

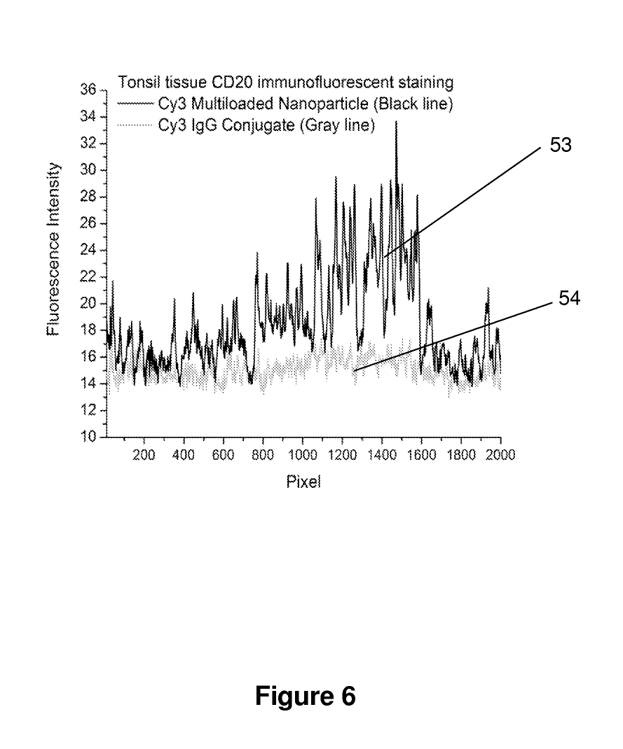

Further, FIG. 6 shows the signal intensity profiles from 53 and 54 in the two images of FIGS. 5A and 5B, indicating that the signal intensity from multi-loaded nanoparticles is higher than that from traditional antibody conjugates across the line profile. The dynamic range of signals from multi-loaded nanoparticles is also greater than traditional antibody conjugates, allowing for more accurate analysis on high and low pixels, indicating the differentiation in the levels of biomarker expression.

### Example 2

### Methods of Synthesis with Chelates

The following is a sample protocol for the preparation of a targeted, multi-loaded lipid-based nanoparticle carrying gadolinium chelates through multiple modes of association:

(1.) Lipid Vesicle with Probes Preparation
a. Exemplary lipid composition:

| | | Molar Percentage |
|---|---|---|
| Component | Abbreviation | in Composition |
| 1,2-distearoyl-sn-glycero-3- | DSPC | 40-60 |
| phosphocholine | | |
| Cholesterol | Chol | 10-40 |
| 1,2-distearoyl-sn-glycero-3- | mPEG₂₀₀₀- | 0.1-10 |
| phosphoethanolamine-N- | DSPE | |
| [methoxy(polyethylene glycol)- | | |
| 2000] | | |
| 1,2-dimyristoyl-sn-glycero-3- | 14:0 PE- | 3-25 |
| phosphoethanolamine-N- | DTPA(Gd) | |
| diethylenetriaminepentaacetic acid | | |
| (gadolinium salt) | | |
| 1,2-distearoyl-sn-glycero-3- | maleimide- | 0.05-5 |
| phosphoethanolamine-N- | PEG₂₀₀₀-DSPE | |
| [maleimide(polyethylene | | |
| glycol)2000] | | |

Note:
The molar percentage in composition can vary interdependently and the sum should remain 100. 14:0 PE-DTPA(Gd) serves the conjugated reporters in which the gadolinium chelate is covalently linked to the lipid portion that is anchored in the lipid layers.

b. Exemplary lipid concentration: 30 mg/ml
c. Add ˜10 ml chloroform, ˜1 ml methanol, and ˜1 ml MilliQ water
d. Vortex to mix
e. Use a rotary evaporator to remove solvent
f. Vacuum for ˜1 hr
g. Freeze-dry ˜460 mTorr ˜−56° C. for 1 hr 30 min
h. Buffer: Freshly prepared 5 mM HEPES or 5 mM phosphate, 0.5 mM gadobenate dimeglumine (Gd-BOPTA, Multihance®), 135 mM NaCl, pH 7.4 Note: Gadobenate dimeglumine serves the encapsulated reporters in the aqueous interior of liposomes.
i. Add 10 ml of buffer to the dried lipid film
j. Thaw at 60° C. and freeze on dry ice. Freeze-thaw multiple times.
k. Extrude through a 80, 100, or 200 nm polycarbonate membrane at ˜60° C. 11-18 times by an extruder
l. Seal in high purity nitrogen and store at 4° C.

### (2.) Reduction of Antibody

a. Prepare 50 mM dithiothreitol (DTT) reducing agent to produce reactive groups in antibodies.
   Note: Other useful agents for reducing antibodies include tris(2-carboxyethyl) phosphine (TCEP), mercaptoethylamine (MEA), cystamine, 2-mercaptoethanol (BME), and so on. The choice of reducing agents depends on the chemical properties of antibodies, the desired fragments of antibodies, the scheme used for fragmentation, and the ease of purification after reactions.
b. Exemplary antibody: Goat anti-rabbit IgG (GAR)
c. Reduce GAR by adding 100 ul 50 mM DTT to 400 μl GAR and incubate at 37° C. for 30 min
d. Transfer to ice water bath and desalt by spin columns
e. Seal in high purity nitrogen and store at 4° C.

### (3.) Preparation of Targeted Multi-Loaded Liposomes

a. Add ˜500 ul reduced antibody to ˜575 ul lipid vesicles containing surface conjugated DTPA(Gd), encapsulated reporters (gadobenate dimeglumine).
b. Vortex
c. Incubate at RT for several hours
d. Seal in high purity nitrogen and store at 4° C.

The above steps are carried out in order in this example, and illustrate a protocol for attaching a modified antibody to a lipid whereby the antibody fragment is displayed on the outer surface of the nanoparticle and can be present in sufficient concentration (numbers of antibody fragments) to cause effective targeting of the nanoparticle, in this case to rabbit IgG.

### Example 3

### Dyes Useful in Encapsulation, Embedding, and Conjugation

Below is a listing of exemplary dyes that can be incorporated into liposomes and micelles via various modes of association.

### Group A—Association by Encapsulation

| Emission | |
|---|---|
| wavelength | Association by |
| (nm) | Encapsulation |
| ~515 nm | carboxyfluorescein |
| ~519 nm | Fluorescein iso- |
| | thiocyanate (FITC) |
| ~565 nm | TAMRA |
| ~576 nm | Phycoerythrin |
| | (PE), Tetramethyl |
| | Rhodamine |
| | Isothiocyanate |
| | (TRITC) |
| ~600 nm | Sulforhodamine B |
| ~660 nm | Allophycocyanin |
| | (APC) |
| ~665 nm | Cy5 |

TAMRA = carboxytetramethylrhodamine
TRITC = tetramethylrhodamine isothiocyanate
The approximate sign “~” is used due to the shift of the emission peaks of the fluorophores under different physical and chemical conditions that can be found in the literature.

### Group B—Association by Embedding

| Emission | |
|---|---|
| wavelength | Association by |
| (nm) | Embedding |
| ~377 nm | Pyrene |
| ~510 nm | BODIPY |
| ~510 nm | DiO |
| ~519 nm | Fluorescein |
| ~534 nm | NBD |
| ~565 nm | DiI |
| ~570 nm | DiIC₁₈(3)-DS |
| ~590 nm | DiA |
| ~601 nm | Texas Red |
| ~665 nm | DiD |
| ~670 nm | DiIC₁₈(5)-DS |
| ~780 nm | DiR |

DiO = 3,3′-dioctadecyloxacarbocyanine perchlorate, DiOC18(3)
DiI = 1,1′-dioctadecyl-3,3,3′,3′-tetramethylindocarbocyanine perchlorate, DiIC18(3)
DiIC₁₈(3)-DS = 1,1′-dioctadecyl-3,3,3′,3′-tetramethylindocarbocyanine-5,5′-disulfonic acid
DiA = 4-(4-(dihexadecylamino)styryl)-N-methylpyridinium iodide, 4-Di-16-ASP
DiD = 1,1′-dioctadecyl-3,3,3′,3′-tetramethylindodicarbocyanine perchlorate, DiIC18(5) oil
DiR = 1,1′-dioctadecyl-3,3,3′,3′-tetramethylindotricarbocyanine iodide, DiIC18(7)
DiIC₁₈(5)-DS = 1′-dioctadecyl-3,3,3′,3′-tetramethylindodicarbocyanine-5,5′-disulfonic acid

For applications in live cells labeling, DiIC₁₈(3)-DS and DiIC₁₈(5)-DS are preferentially used as they carry a net negative charge. It is known that positively charged nanoparticles tend to interact with cells in a non-specific manner and result in high background.

### Group C—Association by Conjugation

| Emission | |
|---|---|
| wavelength | Association by |
| (nm) | Conjugation |
| ~565 nm | TAMRA |
| ~561 nm | Cy3 |
| ~573 nm | Alexa Fluor |
| | 546 |
| ~565 nm | Alexa Fluor |
| | 555 |
| ~576 nm | TRITC |
| ~576 nm | DyLight 549 |
| ~566 nm | HiLyte Fluor |
| | 555 |
| ~665 nm | Cy5 |
| ~665 nm | Alexa Fluor |
| | 647 |
| ~673 nm | DyLight 649 |
| ~675 nm | HiLyte Fluor |
| | 647 |

The above tables only provide exemplary components in each mode of association. Combinations, the permutations thereof, can be generated from the above tables. And many commercially available dyes can be incorporated into the combinations as long as the spectral properties serve the purposes of the nanoparticles.

### Example 4

### Synthesis of a Multiple-Reporter Lipid Nanoparticle

1,1′-dioctadecyl-3,3,3′,3′-tetramethylindocarbocyanine perchlorate (commonly abbreviated as DiI or DiIC18(3)) is a lipophilic tracer and can be embedded in the lipid bilayers of the lipid assemblies. In such construct, it can be coupled with tetramethylrhodamine (TAMRA) as they have similar spectral properties. One can use derivatized TAMRA such as tetramethylrhodamine-5-maleimide and 5-(and -6)-Carboxytetramethylrhodamine, succinimidyl ester (commonly abbreviated as 5(6)-TAMRA, NHS ester, or 5(6)-TAMRA, SE) for conjugation to thiol or amine-derivatized surface of the nanoparticle. 5(6)-TAMRA can be encapsulated in the interior space of the nanoparticle, thus completing multiple mode loading of nanoparticles.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

### REFERENCES

1. Ornatsky, O.; Bandura, D.; Baranov, V.; Nitz, M.; Winnik, M. A.; Tanner, S., Highly multiparametric analysis by mass cytometry. Journal of Immunological Methods 2010, 361, (1-2), 1-20.
2. Bendall, S. C.; Simonds, E. F.; Qiu, P.; Amir, E.-a. D.; Krutzik, P. O.; Finck, R.; Bruggner, R. V.; Melamed, R.; Trejo, A.; Ornatsky, 0.1.; Balderas, R. S.; Plevritis, S. K.; Sachs, K.; Pe'er, D.; Tanner, S. D.; Nolan, G. P., Single-Cell Mass Cytometry of Differential Immune and Drug Responses Across a Human Hematopoietic Continuum. Science 2011, 332, (6030), 687-696.
3. Allen, T. M.; Cullis, P. R., Drug Delivery Systems: Entering the Mainstream. Science 2004, 303, (5665), 1818-1822.
4. Noble, C. O.; Kirpotin, D. B.; Hayes, M. E.; Mamot, C.; Hong, K.; Park, J. W.; Benz, C. C.; Marks, J. D.; Drummond, D.C., Development of ligand-targeted liposomes for cancer therapy. Expert Opinion on Therapeutic Targets 2004, 8, (4), 335-353. 5. Rongen, H. A. H.; Bult, A.; Bennekom, W. P. v., Liposomes and immunoassays. Journal of Immunological Methods 1997, 204, (2), 105-133.
6. Gómez-Hens, A.; Fernández-Romero, J. M., The role of liposomes in analytical processes. Trends in Analytical Chemistry 2005, 24, (1), 9-19.
7. Edwards, K. A.; Baeumner, A. J., Liposomes in analyses. Talanta 2006, 68, (5), 1421-1431
8. Truneh, A.; Machy, P.; Horan, P. K., Antibody-bearing liposomes as multicolor immunofluorescence markers for flow cytometry and imaging. Journal of Immunological Methods 1987, 100, (1-2), 59-71.
9. Singh, A. K.; Cummings, E. B.; Throckmorton, D. J., Fluorescent Liposome Flow Markers for Microscale Particle-Image Velocimetry. Analytical Chemistry 2001, 73, (5), 1057-1061.
10. Ghaghada, K. B.; Ravoori, M.; Sabapathy, D.; Bankson, J.; Kundra, V.; Annapragada, A., New Dual Mode Gadolinium Nanoparticle Contrast Agent for Magnetic Resonance Imaging. PLoS ONE 2009, 4, (10), e7628.
11. Francis C. Szoka, J.; Papahadjopoulos, D., Comparative Properties and Methods of Preparation of Lipid Vesicles (Liposomes). Annual Review of Biophysics and Bioengineering 1980, 9, 467-508.
12. Torchilin, V. P.; Omelyanenko, V. G.; Papisov, M. I.; Bogdanov, A. A., Jr.; Trubetskoy, V. S.; Herron, J. N.; Gentry, C. A., Poly(ethylene glycol) on the liposome surface: on the mechanism of polymer-coated liposome longevity. Biochimica et Biophysica Acta 1994, 1195, (1), 11-20.
13. Torchilin, V. P., Recent Advances with Liposomes as Pharmaceutical Carriers. Nature Reviews Drug Discovery 2005, 4, (2), 145-160.
14. Hermanson, G. T., Bioconjugate Techniques. 2nd ed.; Academic Press: 2008; p 1323.
15. Iyer, A. K.; Khaled, G.; Fang, J.; Maeda, H., Exploiting the enhanced permeability and retention effect for tumor targeting. Drug Discovery Today 2008, 11, (17-18), 812-818.
16. Weng, K. C.; Noble, C. O.; Papahadjopoulos-Sternberg, B.; Chen, F. F.; Drummond, D.C.; Kirpotin, D. B.; Wang, D.; Hom, Y. K.; Hann, B.; Park, J. W., Targeted Tumor Cell Internalization and Imaging of Multifunctional Quantum Dot-Conjugated Immunoliposomes In Vitro and In Vivo. Nano Letters 2008, 8, (9), 2851-2857.

1. A nanoparticle composition comprising:
(a) an organic nanoparticle having an external wall, an inner core region, and optional interior wall portions defining therein additional inner core regions;
(b) targeting molecules linked to said external wall; and
(c) reporter molecules associated with the nanoparticle through at least two of:
(i) encapsulation of the reporter molecules in one or more inner core regions;
(ii) embedding of the reporter molecules in one or more of said external wall and said interior wall portions;
(iii) chemical linkage of the reporter molecules to one or more of said external wall and said interior wall portions;
(iv) specific binding of the reporter molecules to binding partners in one or more of said external wall and said internal wall portions; and
(v) electrostatic binding of the reporter molecules to one or more of said external wall and said interior wall portions.

2. The nanoparticle composition of claim 1 wherein the nanoparticle is either a liposome, a micelle, a multilamellar vesicle, or a multi-vesicular vesicle.

3. The nanoparticle composition of claim 2 wherein the external wall comprises lipids, said lipids being one or more of glycerolipids, phosphatidic acids, phosphatidyl choline, phosphatidyl ethanolamine, phosphatidyl serine, phosphatidyl glycerol, phosphatidyl inositol, sphingolipids, ceramides, sterol lipids, functionalized lipids, cross-linked lipids, and PEGylated lipids.

4. The nanoparticle composition as in claim 1 wherein the reporter molecule is a single species of dye or more than a single species of dye.

5. The nanoparticle composition of claim 4 wherein the dye is at least one of fluorescein, rhodamine, coumarin, BODIPY, Cascade Blue, Pacific Blue, Pacific Orange, NBD, Lucifer Yellow, phycobiliprotein, Texas Red, cyanine, Alexa Fluor, eFluor, DyLight Fluor, or their derivatives.

6. The nanoparticle composition of claim 1 wherein the reporter molecule is a chelating agent, a metal, a chelation complex, a coordination complex, a lanthanide complex, or a metal complex.

7. The nanoparticle composition of claim 1 wherein the reporter molecule is a semiconductor nanocrystal, an enzyme, an enzymatic substrate, a colorimetric substrate, a mass tag, or a Raman tag.

8. The nanoparticle composition of claim 1 wherein the targeting molecule is at least one member selected from the group consisting of an antibody, antibody fragment, antibody mimetic, peptide, nucleotide, aptamer, sugar, glycoprotein, biotin, avidin, streptavidin, NeutrAvidin, CaptAvidin, or folic acid.

9. (canceled)

10. The nanoparticle composition of claim 1 wherein the targeting molecule is linked to said external wall through crosslinking of maleimide and sulfhydryl groups, or crosslinking of amine and carboxyl groups.

11. The nanoparticle composition of claim 1 wherein the reporters are encapsulated within the interior region and conjugated on the internal or external, either or both sides of the walls of the nanoparticle.

12. The nanoparticle composition of claim 1 wherein the reporter molecules are associated with the nanoparticle through at least three of:
(i) encapsulation of the reporter molecules in one or more inner core regions;
(ii) embedding of the reporter molecules in one or more of said external wall and said interior wall portions;
(iii) chemical linkage of the reporter molecules to one or more of said external wall and said interior wall portions;
(iv) specific binding of the reporter molecules to binding partners in one or more of said external wall and said internal wall portions; and
(v) electrostatic binding of the reporter molecules to one or more of said external wall and said interior wall portions.

13. A method of detecting an analyte in a biological sample or test solution comprising adding to the biological sample or test solution a nanoparticle composition comprising:
(a) an organic nanoparticle having an external wall, an inner core region, and optional interior wall portions defining therein additional inner core regions;
(b) targeting molecules linked to said external wall; and
(c) reporter molecules associated with the nanoparticle through at least two of:
(i) encapsulation of the reporter molecules in one or more inner core regions;
(ii) embedding of the reporter molecules in one or more of said external wall and said interior wall portions;
(iii) chemical linkage of the reporter molecules to one or more of said external wall and said interior wall portions;
(iv) specific binding of the reporter molecules to binding partners in one or more of said external wall and said internal wall portions; and
(v) electrostatic binding of the reporter molecules to one or more of said external wall and said interior wall portions;
and detecting the presence of nanoparticles bound to the analyte by the targeting molecule by detecting a signal from the reporter molecule.

14. The method of claim 13 wherein the method of detecting comprises an immunoassay, immunolabeling, immunohistochemistry, immunocytochemistry, Western blotting, dot blotting, flow cytometry, fluorescent activated cell sorting (FACS), bead assays, ELISA, microarrays, capillary electrophoresis, multiplex analysis, chromatography, sensors and microfluidic systems.

15. A method of 13 wherein detecting an antigen on a tissue or a molecular target biological sample comprising contacting said tissue or biological sample with the nanoparticle composition of and detecting the presence of nanoparticles bound to the tissue or biological sample by the targeting molecule by detecting a signal from the reporter molecule.

16. A method of claim 13, comprising imaging or delivering specific effects to a target biological sample, tissue, organ, animal, or human comprising adding to said target the nanoparticle composition of and detecting the presence of nanoparticles by detecting a signal from the reporter molecule or sensitizing the nanoparticles by providing stimulation to the labels.

17. A method of claim 16 wherein the method of detecting comprises magnetic resonance imaging, positron emission tomography, photo acoustic imaging, computed tomography, single-photon emission computed tomography, radio-sensitization, or photo-sensitization.

18. A method for preparing an organic nanoparticle, comprising the steps of:
(a) preparing an organic nanoparticle having an external wall, an inner core region, and, optionally, interior wall portions defining therein additional inner core regions;
(b) attaching a targeting molecule linked to said external wall by coupling said targeting molecule to the external wall; and
(c) incorporating reporter molecules to said nanoparticle through at least two different methods selected from the group consisting of:
(i) encapsulation of reporter molecules in one or more inner core regions;
(ii) embedding of reporter molecules in one or more of said external wall and said interior wall portions;
(iii) chemical linkage of reporter molecules to one or more of said external wall and said interior wall portions;
(iv) specific binding of reporter molecules to binding partners in one or more of said external wall and said interior wall portions; and
(v) electrostatic binding of reporter molecules to one or more of said external wall and said interior wall portions.

19. The method of claim 18 wherein step (a) comprises preparing either a liposome, a micelle, a multilamellar vesicle, or a multi-vesicular vesicle.

20. The method of claim 18 wherein said targeting molecule is an antibody, antibody fragment, peptide, nucleotide, aptamer, biotin, avidin, streptavidin, NeutrAvidin, CaptAvidin, or folic acid linked to said external wall.

21. The method of claim 20 wherein the reporter molecules are selected from the group consisting of a dye, a chelation complex, a coordination complex, an enzyme, an enzymatic substrate, a colorimetric substrate, a semiconductor nanocrystal, a mass tag, and a Raman tag.

22. The method of claim 18 wherein the reporter molecules comprise self-quenching fluorophores, or fluorescence resonance energy transfer (FRET) pairs.
